(19) United States
(12) Patent Application Publication
(10) Pub. No.: US 2024/0040187 A1
(43) Pub. Date: Feb. 1, 2024

(54) Live Broadcast System For Assisting Remote Home Rehabilitation

(71) Applicant: Recovery Plus USA Inc., Middletown, DE (US)

(72) Inventors: Wen Zhou, Middletown, DE (US); Chi Lei, Middletown, DE (US); Yi Fan, Middletown, DE (US); Zhong You, Middletown, DE (US); Xi Chen, Middletown, DE (US); Xiang Zhang, Middletown, DE (US)

(73) Assignee: Recovery Plus USA Inc., Middletown, DE (US)

(21) Appl. No.: 18/072,833

(22) Filed: Dec. 1, 2022

(30) Foreign Application Priority Data
Jul. 27, 2022 (CN) 202210889507.1

(51) Int. Cl. H04N 21/436; H04N 21/422; H04N 21/414; H04N 21/4788

(52) U.S. Cl. CPC H04N 21/436; H04N 21/42201; H04N 21/41407; H04N 21/4788

(57) ABSTRACT

Disclosed is a live broadcast system for assisting remote home rehabilitation. The system includes: an anchor client, a Bluetooth heart rate component, a user client, and a movement video library. The anchor client is configured to display information of a live class, create the live class, start the live class, cancel the live class, interact with a user during a live broadcast, and monitor exercise status of the user and an overview of an exercise status of all users participating in the live class after the live broadcast. The user client is configured to display information of a live class, make an appointment to participate in the live class, enter the live class, provide real-time visual feedback on an exercise mode of a user, display a real-time heart rate of the user, cancel the live class, interact with an anchor during a live broadcast, and display after-class information.

### CROSS REFERENCE TO RELATED APPLICATION

This patent application claims the benefit and priority of Chinese Patent Application No. 202210889507.1, filed on Jul. 27, 2022, the disclosure of which is incorporated by reference herein in its entirety as part of the present application.

### TECHNICAL FIELD

The present disclosure relates to the field of medical rehabilitation, and in particular, to a live broadcast system for assisting remote home rehabilitation.

### BACKGROUND ART

As an important part of modern medicine, rehabilitation, together with prevention, health care, cure, and treatment constitute a unity of modern medicine. A complete medical service is intended not only to treat diseases and save lives, but also to maintain the overall health and provide good quality of life of patients. Rehabilitation is regarded to play an important role in such process. However, the survey shows that many users are still unable to use rehabilitation services due to factors such as time, distance, money, and lack of social support.

The traditional rehabilitation model requires users to go to the hospital for rehabilitation, which causes a lot of inconvenience to the users and their families. In order to obtain rehabilitation services, the users need to spend lots of time and money to travel from home to hospital or rehabilitation center, and those with mobility difficulties may need to be accompanied by their family members. It takes a lot of human and material resources to complete a rehabilitation program. To complete a standardized rehabilitation program means continuous input of time, money, and energy, which makes many users unable to complete it. As a result, the rehabilitation effect cannot meet expectations, and a good quality of life cannot be maintained. Therefore, the traditional rehabilitation model cannot well meet the needs of these users.

The present disclosure aims to assist medical professional to monitor and guide the users to perform rehabilitation exercises in real time through live online rehabilitation class, combined with heart rate feedback and visual feedback, so as to support the users to perform home rehabilitation, and improve their overall health level and quality of life.

### SUMMARY

An objective of the present disclosure is to provide a live broadcast system for assisting remote home rehabilitation, which assists medical professional to monitor and guide the users to perform rehabilitation exercises in real time through live online rehabilitation class, combined with heart rate feedback and visual feedback, so as to support the users to perform home rehabilitation, and improve their overall health level.

In order to achieve the above objective, the present disclosure provides the following technical solutions:

A live broadcast system for assisting remote home rehabilitation is provided, including:

an anchor client, a Bluetooth heart rate component, a user client, and a movement video library.

The anchor client is configured to display information of a live class, create the live class, start the live class, cancel the live class, interact with a user during a live broadcast, and monitor an exercise status of the user and an overview of an exercise status of all users participating in the live class after the live broadcast.

The Bluetooth heart rate component is configured to monitor a heart rate of the user in real time.

The user client is connected to the Bluetooth heart rate component. The user client is configured to display information of a live class, make an appointment to participate in the live class, enter the live class, provide real-time visual feedback of the user, display a real-time heart rate of the user, cancel the live class, interact with an anchor during a live broadcast, and display class history.

The movement video library is configured to store rehabilitation training videos.

Optionally, the live broadcast system may further include: a server. Both the anchor client and the user client may be connected to the server.

The anchor client may further be configured to obtain a video matching the user from the movement video library and send the video to the server, and the server may be configured to recommend the received video matching the user to the user client.

The user client may be configured to send a recorded video to the server, and the server may further be configured to feed back the recorded video by the user to the anchor client.

Optionally, a server may further be connected to the Bluetooth heart rate component. The Bluetooth heart rate component may send the monitored heart rate of the user to the server. The server may be configured to compare the received heart rate of the user with a preset threshold. If the heart rate of the user is not within the preset threshold, a voice prompt may be performed on the user client, and if the heart rate of the user is not within a safety threshold, an alarm prompt may be performed.

Optionally, the anchor client may include a first camera and a first microphone. The first camera may be configured to capture movements of the anchor, and the first microphone may be configured to acquire a voice of the anchor.

Optionally, the user client may include a second camera, and the second camera may be configured to capture movements of the user. Users can see their movements in the live class, and compare their movements with the standard movements. In addition, combined with the real-time guidance of the medical professional, the users can complete standard movements, promoting rehabilitation effects and reducing the risk of exercise injuries.

Optionally, the Bluetooth heart rate component may be a heart rate monitoring device, such as a heart rate belt or a bracelet, which is worn on the user.

According to the specific embodiments provided by the present disclosure, the present disclosure discloses the following technical effects:

The above solution in the present disclosure realizes live streaming by comprehensively utilizing the media capabilities of modern browsers, realizes real-time monitoring of the heart rate of the user and early risk warning in combination with the wearable device, implements online video tutoring, displays movements of the user in real time to give visual feedbacks, and is supplemented by online supervision and interaction of medical professional, so as to guide the user in exercise rehabilitation, thereby easing the restrictions of time, distance, space and economic conditions. The live broadcast system provides users with a remote rehabilitation solution, which is not only conducive to promoting user compliance, improving rehabilitation efficiency, but also promoting exercise safety and medical fairness.

### BRIEF DESCRIPTION OF THE DRAWINGS

To describe the embodiments of the present disclosure or the technical solutions in the related art more clearly, the accompanying drawings required in the embodiments are introduced below. Obviously, the accompanying drawings described below are only some embodiments of the present disclosure. Those of ordinary skill in the art may further obtain other accompanying drawings based on these accompanying drawings without creative labor.

FIG. 1 is an operation flowchart of a live broadcast system for assisting remote home rehabilitation according to an embodiment of the present disclosure; and

FIG. 2 is a schematic diagram of an interaction between a user media stream and an anchor according to the embodiment of the present disclosure.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

An objective of the present disclosure is to provide a live broadcast system for assisting remote rehabilitation, which assists medical professional to monitor and guide the users to perform rehabilitation exercises in real time through live online rehabilitation class, combined with heart rate feedback and visual feedback, so as to support the users to perform home rehabilitation, and improve their overall health level.

To make the above-mentioned objective, features, and advantages of the present disclosure clearer and more comprehensible, the present disclosure will be further described in detail below in conjunction with the accompanying drawings and specific embodiments.

The present disclosure provides a live broadcast system for assisting remote home rehabilitation, including:

an anchor client, a Bluetooth heart rate component, a user client, and a movement video library.

The anchor client is configured to display information of a live class, create the live class, start the live class, cancel the live class, interact with a user during a live broadcast, and monitor an exercise status of the user.

The Bluetooth heart rate component is configured to monitor a heart rate of the user in real time.

The user client is connected to the Bluetooth heart rate component. The user client is configured to display information of a live class, make an appointment to participate in the live class, enter the live class, provide real-time visual feedback on an exercise mode of a user, display a real-time heart rate of the user, cancel the live class, interact with an anchor during a live broadcast, and display after-class information.

The movement video library is configured to store rehabilitation training movement videos.

The anchor client is used by medical professionals, and the user client is used by users receiving rehabilitation services.

The rehabilitation training videos, that is, movement videos, come from the medical database in charge of the medical technical team. Each set of live movement videos has a clear medical purpose. Professional doctors select appropriate movement videos in a targeted manner to meet the medical needs of home rehabilitation training for different disease groups.

As another implementation of the present disclosure, the live broadcast system further includes: a server. Both the anchor client and the user client are connected to the server.

The anchor client is further configured to obtain a video matching the user from the movement videos library and send the video to the server, and the server is configured to recommend the received video matching the user to the user client. That is, medical professional or exercise physiologists can select different movement videos according to different users and different purposes of the live classes.

The user client is configured to send a recorded video to the server, and the server is further configured to feed back the recorded video by the user to the anchor client. That is, when the live broadcast is not performed, the users can record exercise videos according to their own time and send them to medical professional or exercise physiologists for offline guidance.

The user client can be used for exercise feedback of mobile phone users, including the rate of perceived exertion and physical conditions. The server can send the above feedback to the anchor client in real time. The anchor client can perform real-time guidance and communication based on the information of the user client.

As another implementation of the present disclosure, a server is further connected to the Bluetooth heart rate component. The Bluetooth heart rate component sends the monitored heart rate readings of the user to the server. The server is configured to compare the received heart rate readings of the user with a preset threshold. If the heart rate of the user is not within the preset threshold, a voice prompt is performed on the user client, and if the heart rate of the user is not within a safety range, an alarm prompt is performed.

The Bluetooth heart rate component can be a wearable heart rate monitoring device. By wearing the device, users can monitor their heart rate in real time during live class training. The user client analyzes the heart rate data of the user in real time, and gives voice and text prompts for reaching the standard and early warning. Users can control their exercise intensity and reduce exercise risks according to the prompts.

The Bluetooth heart rate component is independently developed for specific Bluetooth heart rate devices and superimposed on the live broadcast page.

As another implementation of the present disclosure, the anchor client may be a mobile phone and specifically includes a first camera and a first microphone. The first camera is configured to capture movements of the anchor, and the first microphone is configured to acquire a voice of the anchor.

The user client may be a mobile phone and specifically includes a second camera. The second camera is configured to capture movements of the user. Users can see their movements in the live class, and compare their movements with the standard movements. In addition, combined with the real-time guidance of the exercise physiologist, the users can complete standard movements, promoting exercise effects and reducing the risk of exercise injuries.

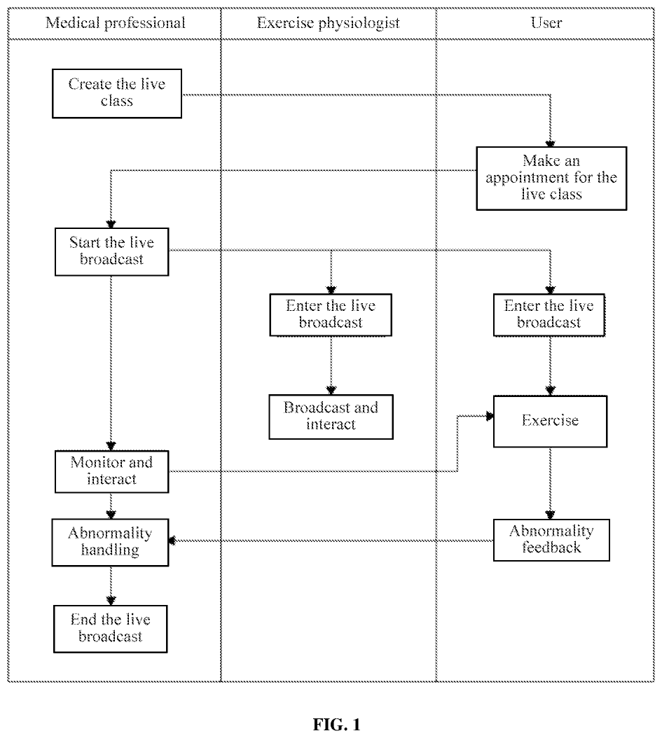

FIG. 1 is an operation flowchart of a live broadcast system for assisting remote home rehabilitation according to an embodiment of the present disclosure. As shown in FIG. 1, first, a live class is created by the medical professional. Second, the user makes an appointment for the live class. The medical professional starts the live broadcast. The exercise physiologist medical professionals and the user enter the live broadcast. The user performs rehabilitation exercises. The exercise physiologist broadcasts, monitor and interacts with the user. The exercise physiologist evaluates whether the movements of the user reach the standard and gives real-time guidance. The medical professional monitors the health status of the user. If it is abnormal, or an urgent information sent by the user is received, they will communicate with the user and handle it in time. The live broadcast will be ended when it is completed.

In addition, the above solution in the present disclosure can be divided into four stages, which are: media capture, media encoding, media delivery, and live communication design.

The media capture is natively supported by the getUserMediaAPI of the Web browser, which is called in the JavaScript program to obtain access to the camera and microphone under the permission of the user.

The specific implementations of the anchor client and the user client are slightly different:

Anchor client: iPhone is used as an external camera and microphone to record MP4 media format (AVC/H.264 video encoding and MP4A audio encoding). This format is widely supported by Web players, and is compatible with desktop and mobile platforms. In addition, it can also recommend the video stream to the user client in a frame (aspect ratio) that matches the mobile phone screen to maximize the presentation of video information.

Media encoding is real-time encoding and slicing of the original media stream captured by the media device, natively supported by the MediaRecorder API of the Web browser, and the encoded media clips are uploaded to the server.

Controllability of Media Quality:

The resolution and frame rate are natively supported by the getUserMedia API.

The encoder and code rate are natively supported by the MediaRecorder API.

The specific implementations of the anchor client and the user client are slightly different:

Anchor client: since iPhone is used as an external camera and microphone, the encoding format is fixed to MP4 (AVC/H.264 video encoding and MP4A audio encoding).

User client: the MP4 or WebM (vp8 video encoding and opus audio encoding) encoding format is automatically selected according to the media capabilities of the user platform, and the anchor client supports the playback of these two formats.

Extensibility:

The anchor client supports the use of multiple iPhones to provide live streams with different encoding qualities, so as to provide suitable picture quality for users with different bandwidths, and can meet low latency through adaptive streaming.

For media delivery, the live server distributes the received anchor stream to the user client, or recommends the video stream of the user to the anchor client.

As the support progress of various browser manufacturers for W3C specifications and IETF Internet standards is not uniform, different streaming media delivery solutions are adopted for different clients without relying on third-party technologies:

Windows/Android/MAC Chrome or Compatible Client:

Media streams are delivered to client players using the Media Source Extensions (MSE) technology.

iPhone/MAC Safari Client:

Media streams are delivered to client players using the HTTP Live Streaming (HLS) protocol.

Live communication design: in addition to solving the necessary links of live streaming such as media capture, media encoding, and media delivery, the present disclosure also needs to solve performance and business requirements such as Network Address Translation (NAT) penetration, high concurrency, low latency, and doctor-patient interaction. For each endpoint accessing the server, the present disclosure designs three WebSocket channels to meet these requirements, which play a key role in the smoothness of the live broadcast process.

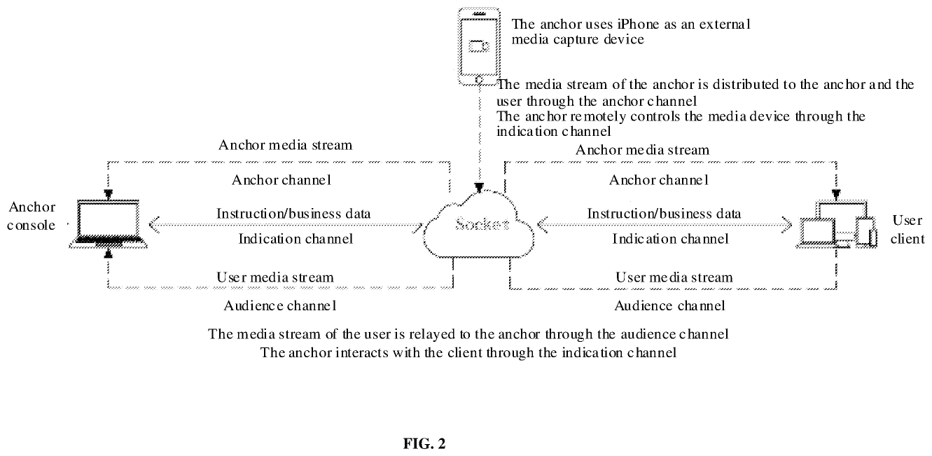

As shown in FIG. 2, from the perspective of users, the three data channels can be divided into: an indication channel, an anchor channel, and an audience channel.

The indication channel is a two-way data channel, mainly used for the interaction of logical instructions.

The anchor channel is a one-way downlink channel dedicated to distributing the anchor media stream to ensure the smoothness of the video stream, through which the anchor console and the user client receive the live video of the anchor.

The audience channel is a one-way upstream channel dedicated to uploading video streams by the user, through which the anchor and medical professional monitor the exercise status of the user.

User client: the access to the camera of the user device is obtained under the permission of the user, so as to facilitate the medical professional to observe the exercise status of the user, ensuring the quality of teaching and preventing risks. The user client supports MAC/iOS Safari version 14.6 and above, or Chrome/Chrome for Android version 76 and above, or a compatible system platform, and automatically selects to acquire MP4 or WebM media formats.

The one-way transmission of media streams using independent channels can ensure the input/output order of stream data, and is conducive to maintaining the buffer queue of media streams. The two-way interactive indication channel conforms to the HTTP request/response model, which can ensure the execution order of instructions and is conducive to maintaining the buffer queue of instructions.

To sum up, the present disclosure combines live broadcast technology with real-time heart rate monitoring, and heart rate feedback can improve the user's perception of exercise intensity and promote exercise safety. The present disclosure provides the user with real-time visual feedback on the exercise mode, which is conducive to timely adjustment of movements and reducing the risk of exercise injuries.

The live broadcast technology involved in the present disclosure is based on the most essential principle of streaming media and relies on the media capabilities of the client browser, and the technical implementation is completely independent and does not depend on any third-party framework or software development kit (SDK).

Each embodiment of the present specification is described in a progressive manner, each embodiment focuses on the difference from other embodiments, and the same and similar parts between the embodiments may refer to each other.

In this specification, some specific examples are used for illustration of the principles and implementations of the present disclosure. The description of the foregoing embodiments is used to help illustrate the method of the present disclosure and the core ideas thereof. In addition, those of ordinary skill in the art can make various modifications in terms of specific implementations and the scope of application in accordance with the ideas of the present disclosure. In conclusion, the content of the present description shall not be construed as limitations to the present disclosure.

What is claimed is:

1. A live broadcast system for assisting remote home rehabilitation, comprising:
an anchor client, a Bluetooth heart rate component, a user client, and a movement video library, wherein
the anchor client is configured to display information of a live class, create the live class, start the live class, cancel the live class, interact with a user during a live broadcast, and monitor an exercise status of the user and an overview of an exercise status of all users participating in the live class after the live broadcast;
the Bluetooth heart rate component is configured to monitor a heart rate of the user in real time;
the user client is connected to the Bluetooth heart rate component, and the user client is configured to display information of a live class, make an appointment to participate in the live class, enter the live class, provide real-time visual feedback on an exercise mode of a user, display a real-time heart rate of the user, cancel the live class, interact with an anchor during a live broadcast, and display after-class information; and the movement video library is configured to store rehabilitation training videos.

2. The live broadcast system for assisting remote home rehabilitation according to claim 1, further comprising: a server, wherein both the anchor client and the user client are connected to the server;
the anchor client is further configured to obtain videos matching the user from the movement video library and send the videos to the server, and the server is configured to recommend the received videos matching the user to the user client; and the user client is configured to send a recorded video to the server, and the server is further configured to feed back the recorded video by the user to the anchor client.

3. The live broadcast system for assisting remote home rehabilitation according to claim 1, wherein a server is further connected to the Bluetooth heart rate component, the Bluetooth heart rate component sends the monitored heart rate of the user to the server, the server is configured to compare the received heart rate of the user with a preset threshold, if the heart rate of the user is not within the preset threshold, a voice prompt is performed on the user client, and if the heart rate of the user is not within a safety threshold, an alarm prompt is performed.

4. The live broadcast system for assisting remote home rehabilitation according to claim 1, wherein the anchor client comprises a first camera and a first microphone, the first camera is configured to capture movements of the anchor, and the first microphone is configured to acquire a voice of the anchor.

5. The live broadcast system for assisting remote home rehabilitation according to claim 1, wherein the user client comprises a second camera, and the second camera is configured to capture movements of the user.

6. The live broadcast system for assisting remote home rehabilitation according to claim 1, wherein the Bluetooth heart rate component is a heart rate monitor, which is worn on the user.
